(19) United States
(12) Patent Application Publication
(10) Pub. No.: US 2002/0032188 A1
(43) Pub. Date: Mar. 14, 2002

(54) Compositions and methods for treatment of anal fissure

(76) Inventor: Azariah Jossifoff, Ramat Gan (IL)

Correspondence Address: DARBY & DARBY, 805 THIRD AVENUE, 27TH FLR., NEW YORK, NY, 10022, US

(21) Appl. No.: 09/904,204

(22) Filed: Jul. 12, 2001

(30) Foreign Application Priority Data
Jul. 12, 2000 (IL) 137275

(51) Int. Cl. A61K 31/554; A61K 31/44; A61K 31/21

(52) U.S. Cl. 514/211.07; 514/509; 514/354

(57) ABSTRACT

There is provided a pharmaceutical composition comprising isosorbide dinitrate, wherein said composition is in the form of an aqueous gel containing not more than about 15 wt.% of a water-soluble lipophilic substance. Optionally, the gel may comprise a calcium channel blocker as an additional active ingredient. A composition comprising diisosorbide dinitrate and nifedipine is also disclosed. There is also provided a method for treating anal fissure, comprising applying to a locus in need of such treatment an efficacious amount of a composition according to the invention.

### FIELD OF THE INVENTION

The present invention concerns gel's or emulsion gels for the treatment of anal fissures.

### BACKGROUND OF THE INVENTION

An anal fissure is a tear in the lining of the anus. Fissures may be superficial and heal rapidly, but often they become chronic. Such fissures are extremely painful and cause marked spasm of the anal sphincter. Anal fissures are associated with pain (especially during bowel movements), bleeding, and/or itching. Stretching of the anus by defecation or examination can cause extreme distress.

The exact cause of anal fissures is at present unknown. However, there is a growing body of evidence which suggests that increased muscle tone (hypertonicity) of the internal anal sphincter is a predisposing factor in the formation of anal fissures, and possibly hemorrhoids. Blood vessels normally supply an adequate blood supply to the site where fissures occur. When sphincter tone is increased, the blood supply to the affected area is decreased and even minimal trauma, such as a hard stool, may increase the susceptibility to a fissure.

Acute anal fissures can often be cured in two to three weeks by adopting measures such as a low residue diet and ingestion of stool softeners. However, about 40% of the cases of anal fissure occurring in the U.S. each year do not respond to such treatment. Such fissures are considered chronic, rather than acute anal fissures. Treatment of chronic anal fissures with persisting spasm has traditionally required more drastic measures such as excision of the fissure (sphincterotomy with excision of the sentinel pile). This procedure is known as lateral internal anal sphincterotomy. In this procedure, the internal anal sphincter is partially excised. Severing the internal anal sphincter has been shown to promote the healing of a large percentage of chronic fissures. However, this procedure has also been found to create some form of fecal incontinence in up to 35% of patients receiving this surgical treatment.

The use of nitric oxide (NO) donors, such as nitroglycerine or isosorbide dinitrate (ISDN), has been known for years for the treatment of angina pectoris and hypertension. More recently, the topical application of compositions containing a single active component either of the nitric oxide donor type e.g., nitroglycerin or ISDN or of the calcium channel blocker type, e.g. diltiazem or nifedipine, have been reported in the medical literature as providing relief from anal fissures, particularly of the acute type. Nitric oxide is regarded as a neurotransmitter mediating vasodilation. Presumably, NO acts by bringing about anal sphincter relaxation. However, with chronic fissures the results hitherto have been less encouraging: the treatment failed to heal the ailment and/or caused undesirable side effects, such as headaches.

Thus, Gorfine, S.R., in a communication to The New England Journal of Medicine, Vol. 333, No. 17, pp. 1156-7, October, 1995, reported that treatment with an ointment containing 0.3% nitroglycerin provided rapid relief of anal pain and promoted the healing of anal fissures and ulcers. Schouten, W. R., et al., Gut 1996 Sep; 39(3): 465-9, reported similar results for the treatment of chronic anal fissure with ISDN. Lysy, J., et al., Dis Colon Rectum, 1998, Nov;41(11):1406-10, also reported on the treatment of chronic anal fissure with ISDN. They concluded that topical ISDN is an effective treatment for chronic anal fissure. Antropoli, C., et al., Dis Colon Rectum, 1999, Aug;42(8): 1011-5, reported on total remission from acute anal fissure after 21 days of therapy in 95% of 141 patients after treatment with topical 0.2% nifedipine gel every twelve hours for the aforementioned period. On the other hand, Hyman, N. H., et al., Dis Colon Rectum, 1999, Mar;42(3): 383-5, conducted a study of the long term usefulness of nitroglycerin treatment for anal fissures, by topical application of a 0.3% ointment. They concluded that topical nitroglycerin was only effective in approximately one half of the patients with anal fissure. Moreover, there was a very high incidence of adverse reactions such as headaches.

An additional drawback to the methods used in the prior art for the application of NO donors to the anal area is due to the way in which NO donors are typically packaged. Compounds which function as NO donors generally comprise one or more nitro groups (—NO₂), which tend to make the compound unstable. For this reason, commercially available NO donors are usually packaged in dilute form, so as to minimize the risk of hazardous explosion during transport and handling. Thus, for example, ISDN is available commercially in the form of a powder containing from 40-60 wt.% lactose or maltose. Although the presence of lactose does not inhibit the preparation of oily formulations such as ointments or creams for the topical delivery of ISDN, the presence of lactose prevents the incorporation of ISDN into a water-based gel formulation, since the lactose leads to the formation of inhomogeneities in the composition. However, because non-aqueous-based NO-donor formulations for the treatment of anal fissures may permanently stain the patient's clothing around the gluteal area, it would be desirable to be able to provide an NO-donor containing water-based gel, which would not permanently stain the patient's clothing.

### SUMMARY OF THE INVENTION

It would be desirable to provide a non-surgical method for the treatment of anal fissures, particularly chronic anal fissures, which method is more efficacious than methods presently available and which does not suffer from the drawbacks of the presently available non-surgical methods for the treatment of anal fissures.

There is thus provided, in accordance with a preferred embodiment of the invention, a pharmaceutical composition comprising isosorbide dinitrate, wherein said composition is in the form of an aqueous gel containing not more than about 15 wt.% of a water-soluble lipophilic substance.

In a preferred embodiment of the invention, the isosorbide dinitrate is present in an amount between about 0.1 and about 0.4 wt.%, more preferably about 0.2 wt.%.

In a preferred embodiment of the invention, the water soluble lipophilic substance is selected from the group consisting of polyethylene glycol and polypropylene glycol.

In a preferred embodiment of the invention, the gel contains dimethylsulfoxide.

In a preferred embodiment of the invention, the composition further comprises a second active ingredient which is a vasodilator. In a preferred embodiment of the invention, the vasodilator is a calcium channel blocker. In a preferred embodiment of the invention, the calcium channel blocker is selected from the group consisting of nifedipine and diltiazem, In an especially preferred embodiment of the invention, the calcium channel blocker is nifedipine. In a preferred embodiment of the invention, when the composition includes a calcium channel blocker, the isosorbide dinitrate is present in an amount between about 0.1 and about 0.4 wt.%, more preferably about 0.2 wt.%, and the calcium channel blocker is present in an amount between about 0.1 and about 0.4 wt.%, more preferably about 0.2 wt.%.

There is also provided, in a preferred embodiment of the invention, a pharmaceutical composition for the treatment of anal fissures, comprising isosorbide dinitrate and nifedipine and at least one pharmaceutically acceptable excipient, carrier or diluent therefor, and wherein said composition is in a topically administrable form. In a preferred embodiment of the invention, the pharmaceutical composition is in the form of a water-based gel containing not more than about 15 wt.% of a water soluble lipophilic substance. In a preferred embodiment of the invention, the isosorbide dinitrate is present in an amount between about 0.1 and about 0.4 wt.%. In a preferred embodiment of the invention, the nifedipine is present in an amount between about 0.1 and about 0.4 wt.%.

There is also provided in accordance with a preferred embodiment of the invention a method for treating anal fissure, comprising applying to an affected locus of a patient suffering from anal fissure an efficacious amount of a pharmaceutical composition comprising isosorbide dinitrate, wherein said composition is in the form of an aqueous gel containing not more than about 15 wt.% of a water-soluble lipophilic substance. In a preferred embodiment of the invention, the isosorbide dinitrate is present in an amount between about 0.1 and about 0.4 wt.%. In an especially preferred embodiment of the invention, the isosorbide dinitrate is present in an amount of about 0.2 wt.%. In a preferred embodiment of the invention, the water-soluble lipophilic substance is selected from the group consisting of polyethylene glycol and propylene glycol. In a preferred embodiment of the invention, the composition comprises dimethylsulfoxide. In a preferred embodiment of the invention, the composition further comprises a calcium channel blocker. In a preferred embodiment of the invention, calcium channel blocker is selected from the group consisting of nifedipine and diltiazem. In an especially preferred embodiment of the invention, the calcium channel blocker is nifedipine. In a preferred embodiment of the invention, the isosorbide dinitrate is present in an amount between about 0.1 and about 0.4 wt.%. In an especially preferred embodiment of the invention, the isosorbide dinitrate is present in an amount of about 0.2 wt.%. In a preferred embodiment of the invention, nifedipine is present in an amount between about 0.1 and about 0.4 wt.%. In an especially preferred embodiment of the invention, nifedipine is present in an amount of about 0.2 wt.%. In a preferred embodiment of the invention, the anal fissure is acute anal fissure. In another preferred embodiment of the invention, the anal fissure is chronic anal fissure. In a preferred embodiment of the invention, the isosorbide dinitrate constitutes a first active ingredient, the calcium channel blocker constitutes a second active ingredient, and the isosorbide dinitrate and the calcium channel blocker are each present in an amount which, if administered alone, is not efficacious in the treatment of anal fissure but which when administered together with the other active ingredient in the amount present in the composition is efficacious in the treatment of anal fissure.

There is also provided in accordance with a preferred embodiment of the invention a method for treatment of anal fissure, comprising applying to an affected locus of a patient suffering from anal fissure a pharmaceutical composition comprising isosorbide dinitrate and nifedipine and at least one pharmaceutically acceptable excipient, carrier or diluent therefor, and wherein said composition is in a topically administrable form. In a preferred embodiment of the invention, the anal fissure is acute anal fissure. In a preferred embodiment of the invention, the anal fissure is chronic anal fissure. In a preferred embodiment of the invention, the isosorbide dinitrate constitutes a first active ingredient, the nifedipine constitutes a second active ingredient, and the isosorbide dinitrate and the nifedipine are each present in an amount which, if administered alone, would not be efficacious in the treatment of anal fissure but which when administered together with the other active ingredient in the amount present in the composition is efficacious in the treatment of anal fissure.

There is also provided in accordance with a preferred embodiment of the invention a method of preparing an aqueous gel pharmaceutical composition comprising isosorbide dinitrate, the method comprising the step of dissolving the isosorbide dinitrate in dimethylsulfoxide to form a first solution and subsequently dissolving said first solution in a first aqueous solvent, whereby to form said aqueous gel. In a preferred embodiment of the invention, the first aqueous solvent contains an additional pharmaceutically active agent dissolved therein. In a preferred embodiment of the invention, the additional pharmaceutically active agent is a calcium channel blocker. In a preferred embodiment of the invention, the calcium channel blocker is nifedipine. In a preferred embodiment of the invention, the additional pharmaceutically active agent has been dissolved in a second aqueous solvent to form a second solution and said second solution has then been mixed into said first aqueous solvent prior to addition of said first solution thereto. In a preferred embodiment of the invention, the said first aqueous solvent comprises up to 15 wt.% of a water-soluble lipophilic substance dissolved therein. In a preferred embodiment of the invention, the water-soluble lipophilic substance is selected from the group consisting of polyethylene glycol 400 and propylene glycol. In a preferred embodiment of the invention, the isosorbide dinitrate is present at a concentration between about 0.1 and about 0.4 wt.% of the gel formed. In an especially preferred embodiment of the invention, the isosorbide dinitrate is present at a concentration of about 0.2 wt.% of the gel formed. In a preferred embodiment of the invention, the isosorbide dinitrate is present at a concentration between about 0.1 and about 0.4 wt.% of the gel formed, and the additional pharmaceutically acceptable agent is present at a concentration between about 0.1 and about 0.4 wt.% of the gel formed. In an especially preferred embodiment of the invention, the isosorbide dinitrate is present at a concentration of about 0.2 wt.% of the gel formed, and said additional pharmaceutically acceptable agent is present at a concentration of about 0.2 wt.% of the gel formed. In a preferred embodiment of the invention, the additional pharmaceutically active agent is nifedipine.

There is also provided in accordance with a preferred embodiment of the invention a method for treating anal fissure, comprising applying to an affected locus of a patient suffering from anal fissure an efficacious amount of a combination of isosorbide dinitrate (ISDN) and a calcium channel blocker (CCB). In a preferred embodiment of the invention, the ISDN and the CCB are provided in separate pharmaceutical preparations and are applied separately to said locus. In one preferred embodiment of the invention, the calcium channel blocker is diltiazem. In another preferred embodiment of the invention, the calcium channel blocker is nifedipine. In one preferred embodiment of the invention, the ISDN and said CCB are applied simultaneously. In another preferred embodiment of the invention, the ISDN and said CCB not applied simultaneously and an efficacious amount of each pharmaceutical preparation is applied to said locus. In a preferred embodiment of the invention, the ISDN is applied prior to defecation, preferably less than 30 minutes prior to defecation, more preferably less than 20 minutes prior to defecation, and said CCB is applied two to four times a day without reference to defecation. In a preferred embodiment of the invention, the separate pharmaceutical compositions are both gels.

### DETAILED DESCRIPTION OF PREFERRED EMBODIMENTS

In one aspect, the present invention concerns a pharmaceutical composition, a method for making the composition, and a method for treating anal fissure, particularly chronic anal fissure, using such a composition, wherein said composition is in the form of a water-based (aqueous) gel comprising isosorbide dinitrate. Prior to the present invention, such a gel was not known in the art, and its preparation was not obvious to the skilled artisan. In a preferred embodiment of the present invention, the composition comprises a second active ingredient useful in the treatment of anal fissure such as a calcium channel blocker or other vasodilator. Calcium channel blockers, such as nifedipine and diltiazem, are a particularly preferred group of second active ingredients. Nifedipine, which has been used in the treatment of heart disease and hypertension for many years, is especially preferred as a second active ingredient.

One of the advantages of water-based gels, such as those of the present invention, is that unlike the topically administrable ointments and creams known in the art for the treatment of anal fissure, water-based gels do not stain patients' clothes or undergarments upon coming into contact with them. This can help patients avoid embarrassing stains from appearing on their clothes as a result of treatment of anal fissure using a topical medicament. The water-based gels of the present invention tend to be smooth, elegant and produce cooling effects because of the evaporation of the water. Such gels may also dry out to form films that adhere well to the skin and that can easily be removed by washing, as desired. The water-based gels of the present invention may also include in addition to water one or more additional appropriate water-soluble volatile solvents, such as alcohols like ethanol or polyols like propylene glycol, provided that these are present in amounts which allow the gel to maintain its advantageous properties as an aqueous gel.

As explained above, compounds which function as nitric oxide (NO) donors generally comprise one or more nitro groups (—NO₂), which tend to make these compounds unstable. For this reason, commercially available NO-donating compounds are usually packaged in dilute form, so as to minimize the risk of hazardous explosion during transport and handling. Thus, for example, ISDN is available commercially in the form of a powder containing from 40-60 wt.% lactose or maltose. Although the presence of lactose does not inhibit the preparation of oily formulations such as ointments or creams for the topical delivery of ISDN, the presence of lactose prevents the incorporation of ISDN into a water-based gel formulation, since the lactose leads to the formation of inhomogeneities in the composition.

Thus, an important factor in the development of the present invention was the surprising and non-obvious discovery that dimethylsulfoxide (DMSO) can act as a solvent for commercially available ISDN packaged in lactose. The resulting DMSO-based solution is itself water-soluble, and thus enables incorporation of ISDN into a water-based gel. Typically, the ratio of ISDN/lactose powder to DMSO used to prepare the DMSO-based solution will be in the range of from about 30 g ISDN/lactose powder mixture per 10 ml DMSO to about 60 g ISDN/lactose powder mixture per 10 ml DMSO. Preferably, the amount of DMSO used will be the minimum amount required to dissolve to a clear solution the ISDN/lactose needed to prepare the final preparation. Thus, for example, if it is desired to prepare 1 kg of 0.2 wt.% ISDN gel, about 40 g of ISDN/lactose powder mixture are required. The minimum amount of DMSO required to dissolve the ISDN/lactose mixture is about 10 ml. The amount of DMSO-based solution used in the preparation of the gel will accordingly vary in relation to the total percentage of ISDN to be incorporated into the gel and degree of dilution of ISDN in lactose prior to dissolution of the ISDN/lactose powder mixture in DMSO.

It will be appreciated by skilled artisans not only that identification of DSMO as a material capable of functioning as both a solvent and solute was non-trivial, but that finding the proper proportion of DMSO to use relative to the other ingredients was non-trivial as well. This is because DMSO is well-known in the art as a material used to increase absorption of drugs into the skin (i.e. it acts as an accelerant, see e.g. Lachman et al., “The Theory and Practice of Industrial Pharmacy”: “They appear to swell the stratum corneum and leach out essential structural material, thus reducing the diffusional resistance and increasing the permeability.”). In the practice of the present invention, however, where it is desired to achieve a local and not systemic effect, increasing the permeability of the skin to ISDN and/or another active agent can lead to such undesired systemic effects, including the unwanted side-effects known to in the art to accompany systemic administration of ISDN, calcium channel blockers and other pharmaceutically active ingredients which can be used in the practice of the present invention. Furthermore, high concentrations of DMSO are known in the art ot cause skin irritation, including itching. Thus the choice of DMSO per se, as well as the particular proportions in which to use DMSO in the practice of the present invention, were not obvious choices to the skilled artisan at the time the present invention was made.

In a preferred embodiment of the invention, the ISDN is present in a concentration between about 0.1 and about 0.4 wt.%, more preferably about 0.2 wt.%. Preferably the ISDN will be present with a second active ingredient, and the ISDN and second active ingredient will each independently be present in a concentration between about 0.1 wt.% and about 0.4 wt.%. An especially preferred concentration for both the ISDN and the second active ingredient is about 0.2 wt.%.

Within the scope of the present invention are contemplated aqueous gel compositions comprising up to about 15 wt.% of a water-soluble lipophilic substance, such as polyethylene glycol and propylene glycol. Although in principle higher concentrations of the water-soluble lipophilic substance can be used, increasing the concentration of the water-soluble lipophilic substance leads to loss of the advantageous properties of the gel and makes the gel more emulsion-like. The water-soluble lipophilic substance may aid in the solubulization of the ISDN and/or the second active ingredient. The incorporation of the water-soluble lipophilic substance facilitates the solubilization of active ingredients that may be primarily oil soluble in a vehicle that maintains for the most part the advantageous properties of a simple aqueous gel with the water-soluble lipophilic substance, previously delineated above.

In a preferred embodiment of the invention, the concentration of ISDN incorporated into the gel of the invention will be efficacious for treatment of anal fissure. However, as will be explained below, the present invention in another aspect concerns the discovery that ISDN and other active ingredients, preferably active ingredients known to have therapeutic value in the treatment of anal fissure, particularly calcium channel blockers and especially nifedipine, can be used in a complementary fashion treat two parameters in the pathology of anal fissure, and may even be used synergistically. Thus, in a preferred embodiment of the invention, the water-based gel of the present invention may contain both ISDN and, for example nifedipine. In a preferred embodiment of the invention, the ISDN is present in a concentration that alone is not efficacious for the treatment of anal fissure, and the second active ingredient, such as nifedipine, is also present in a concentration that alone is not efficacious for the treatment of anal fissure, but which ingredients together are present in a concentration efficacious in the treatment of anal fissure. Preferred concentration ranges for both the ISDN and additional ingredient, each independently, are about 0.1 to about 0.4 wt.%.

Also contemplated within the scope of the present invention is a method of treatment of anal fissure, particularly chronic anal fissure, comprising applying to an affected locus of a patient suffering from anal fissure a composition according to the present invention. Application of the composition to the affected locus will typically be made two or three times a day for a period of 10 to 21 days. Preferably, application of the composition is made three times a day for 21 days.

In another aspect, the present invention concerns pharmaceutical compositions for topical administration (not necessarily in aqueous gel form) comprising a synergistic combination of ISDN and nifedipine. Because synergistic combinations enable the active ingredients to be used in smaller amounts than each component individually would have to be used in order for the composition to be efficacious (i.e., less than half the amount required for each component individually to be effective), use of the synergistic combination of the present invention may results in fewer or substantially no occurrences of the undesirable side effects encountered in treatments using a single active substance (e.g., headaches). No detrimental systemic side effects were observed in patients treated with an integrated synergistic preparation comprising ISDN and nifedipine, unlike in cases where patients were treated with preparations based on nitroglycerin alone.

Such synergistic pharmaceutical compositions for topical administration comprise, in addition to the active ingredients, at least one pharmaceutically acceptable excipient, diluent or carrier therefor, such as are known in the art. Such compositions may be in the form of pastes, creams, ointments, lotions, jellies, salves, gels, and other topically administrable forms of compositions, as are well known in the art. If the composition is not homogeneous (i.e. it is not uniphasic and the ISDN and nifedine are present as particles rather than in solution), the size of the particles of the active ingredients, ISDN and nifedipine, should be sufficiently large to ensure a primarily local effect of the ingredients and avoid significant systemic effects, but sufficiently small to enable penetration of the active ingredients into the area to be treated. If the composition is in the form of a gel in which both ISDN and nifedipine have been dissolved, then lower concentrations of these agents may be employed than in other types of compositions in which ISDN and nifedipine are present as particles.

The following illustrative and non-limitative examples are intended to help the skilled artisan better understand how the present invention may be performed.

### EXAMPLE 1

### Preparation of an Emulsion Gel Containing 0.2% ISDN and 0.2% Nifedipine

The following is an example of how a 1000 gram batch of a typical emulsion gel containing 0.2% isosorbide dinitrate and 0.2% nifedipine was prepared. The steps were carried out under lighting conditions which did not cause degradation of the nifedipine.

(1) A gel was prepared containing 2 g Carbopol (a mixture of carbomers), 1 g ethylenediaminetetraacetic acid (EDTA), 2 g Nipagin (propylparaben, an antimicrobial preservative) and 18.5 g triethanolamine made up with water to a total mass of 850 grams.

(2) Two grams of nifedipine were initially mixed into 10 ml of 96% ethanol. To this there was subsequently added 40 grams of PEG (polyethyleneglycol)-400 to yield a solution. After vigorous stirring, 85 grams of propylene glycol were added to the solution.

(3) Five grams of a mixture containing 40 wt.% isosorbide dinitrate (ISDN) and 60 wt.% lactose were weighed into a separate vessel. To this were added 10 grams of dimethyl sulfoxide (DMSO). This mixture was stirred until a clear solution was obtained.

(4) The ISDN solution was added to the nifedipine solution.

(5) The combined solution (ISDN+nifedipine) was introduced with rapid stirring to the gel of step (1) above, to form a product gel.

(6) The product gel so obtained is an emulsion gel containing 0.2 wt.% of each of the active ingredients, i.e. ISDN and nifedipine.

### EXAMPLE 2

### Test Results of Emulsion Gel Containing 0.2% ISDN and 0.2% Nifedipine on Patients Suffering from Chronic Anal Fissure

Two hundred tubes each containing about 50 grams of the emulsion gel product of Example 1 were prepared and distributed for treatment to patients suffering from chronic anal fissure. Two hundred patients were instructed to apply the gel 3-4 time daily immediately before bowel movement for 21 to 45 days. From the reports of the physicians accompanying this test and the patients receiving treatment, it was apparent that in cases of chronic fissures, the alleviation of symptoms and healing were superior to that observed when treatment involved only one of the active components alone. Essentially no detrimental side effects were observed.

Most of the treatments of chronic conditions began with a gel containing only nifedipine. In about 40% of the cases in which there was improvement but healing was not complete, the patient was transferred to treatment with the integrated gel. In 90% of these cases, the problem was completely alleviated.

### EXAMPLE 3

### Comparative Example—Attempts to Prepare Water-soluble Solutions of ISDN

Various solvents were used in an attempt to obtain a water-soluble solution of commercially available ISDN from a mixture of 40 wt.% ISDN and 60 wt.% lactose.

(a) Attempts to the dissolve the ISDN/lactose mixture in various proportions of propylene glycol were unsuccessful—a precipitate formed.

(b) Attempts to the dissolve the ISDN/lactose mixture in various proportions of PEG-400 were unsuccessful—a precipitate formed, even under conditions of gentle heating (i.e. ˜40° C.).

(c) Dissolution of 1 part ISDN/lactose mixture in 10 parts water (by weight) containing 10 wt.% PEG-400 and 10 wt.% propylene glycol under gentle heating (˜40° C.) resulted in a suspension from which material precipitated almost immediately.

(d) Dissolution of 1 part ISDN/lactose mixture in 5 or 10 parts (by weight) of 96% ethyl alcohol with gentle heating (˜40° C.) to yield an initial solution, followed by addition of 20 parts water by weight for each part by weight of the initial solution to yield a secondary solution, resulted in the rapid formation of crystals in the secondary solution.

(e) Attempts to dissolve ISDN/lactose mixture in acetone in various proportions resulted in dissolution of the ISDN but not the lactose.

(f) Dissolution of ISDN/lactose mixture in chloroform in various proportions resulted in separation of the aqueous and organic phases into, respectively, lower and upper layers.

Many modifications and variations that do not deviate from the basic spirit of the present invention are possible and will be obvious to one skilled in the art. For example, effective combinations of the active ingredients of the present invention can also be formulated as semi-solid topical preparations, such as a cream or an ointment. Standard texts such as Remington's, Practice of Pharmacy and the Pharmaceutical Codex herewith included herein by reference provide methods and procedures by which various compositions for general or specific purposes may be prepared and used. It is therefore to be understood that the present invention may be practiced with various modifications that do not deviate from its basic spirit.

1. A pharmaceutical composition comprising as a first active ingredient isosorbide dinitrate, wherein said composition is in the form of an aqueous gel containing not more than about 15 wt.% of a water-soluble lipophilic substance.

2. A pharmaceutical composition according to claim 1, wherein the isosorbide dinitrate is present in an amount between about 0.1 and about 0.4 wt.%.

3. A pharmaceutical composition according to claim 2, wherein the isosorbide dinitrate is present in an amount of about 0.2 wt.%.

4. A composition according to claim 1, wherein the water soluble lipophilic substance is selected from the group consisting of polyethylene glycol and polypropylene glycol.

5. A composition according to claim 4, wherein the gel contains dimethylsulfoxide.

7. A composition according to claim 1, wherein the composition further comprises a second active ingredient which is a vasodilator.

8. A composition according to claim 7, wherein the vasodilator is a calcium channel blocker.

9. A composition according to claim 8, wherein said calcium channel blocker is selected from the group consisting of nifedipine and diltiazem.

10. A composition according to claim 9, wherein said calcium channel blocker is nifedipine.

11. A pharmaceutical composition according to claim 7, wherein the isosorbide dinitrate is present in an amount between about 0.1 and about 0.4 wt.%.

12. A pharmaceutical composition according to claim 11, wherein the isosorbide dinitrate is present in an amount of about 0.2 wt.%.

13. A pharmaceutical composition according to claim 7, wherein the calcium channel blocker is present in an amount between about 0.1 and about 4.0 wt.%.

14. A pharmaceutical composition according to claim 13, wherein the calcium channel blocker is nifedipine and is present in an amount of about 0.2 wt.%.

16. A pharmaceutical composition for the treatment of anal fissures, comprising isosorbide dinitrate and nifedipine and at least one pharmaceutically acceptable excipient, carrier or diluent therefor, and wherein said composition is in a topically administrable form.

17. A pharmaceutical composition according claim 16, wherein said composition is in the form of a water-based gel containing not more than about 15 wt.% of a water soluble lipophilic substance.

18. A pharmaceutical composition according to claim 16, wherein the isosorbide dinitrate is present in an amount between about 0.1 and about 0.4 wt.%.

19. A pharmaceutical composition according to claim 16, wherein the nifedipine is present in an amount between about 0.1 and about 0.4 wt.%.

20. A method of treatment of anal fissure, comprising applying to an affected locus of a patient suffering from anal fissure an efficacious amount of a pharmaceutical composition comprising isosorbide dinitrate, wherein said composition is in the form of an aqueous gel containing not more than about 15 wt.% of a water-soluble lipophilic substance.

21. A method according to claim 20, wherein the isosorbide dinitrate is present in an amount between about 0.1 and about 0.4 wt.%.

22. A method according to claim 21, wherein the isosorbide dinitrate is present in an amount of about 0.2 wt.%.

23. A method according to claim 20, wherein the water-soluble lipophilic substance is selected from the group consisting of polyethylene glycol and propylene glycol.

24. A method according to claim 23, wherein the composition comprises dimethylsulfoxide.

25. A method according to claim 20, wherein the composition further comprises a calcium channel blocker.

26. A method according to claim 25, wherein said calcium channel blocker is selected from the group consisting of nifedipine and diltiazem.

27. A method according to claim 26, wherein said calcium channel blocker is nifedipine.

28. A method according to claim 27, wherein the isosorbide dinitrate is present in an amount between about 0.1 and about 0.4 wt.%.

29. A method according to claim 28, wherein the isosorbide dinitrate is present in an amount of about 0.2 wt.%.

30. A method according to claim 27, wherein the nifedipine is present in an amount between about 0.1 and about 0.4 wt.%.

31. A method according to claim 30, wherein the nifedipine is present in an amount of about 0.2 wt.%.

32. A method according to claim 20, wherein said anal fissure is acute anal fissure.

33. A method according to claim 20, wherein said anal fissure is chronic anal fissure.

34. A method according to claim 25, wherein the isosorbide dinitrate constitutes a first active ingredient, the calcium channel blocker constitutes a second active ingredient, and the isosorbide dinitrate and the calcium channel blocker are each present in an amount which, if administered alone, is not efficacious in the treatment of anal fissure but which when administered together with the other active ingredient in the amount present in the composition is efficacious in the treatment of anal fissure.

35. A method for the treatment of anal fissure, comprising applying to an affected locus of a patient suffering from anal fissure a pharmaceutical composition comprising isosorbide dinitrate and nifedipine and at least one pharmaceutically acceptable excipient, carrier or diluent therefor, and wherein said composition is in a topically administrable form.

36. A method according to claim 35, wherein said anal fissure is acute anal fissure.

37. A method according to claim 35, wherein said anal fissure is chronic anal fissure.

38. A method according to claim 35, wherein the isosorbide dinitrate constitutes a first active ingredient, the nifedipine constitutes a second active ingredient, and the isosorbide dinitrate and the nifedipine are each present in an amount which, if administered alone, would not be efficacious in the treatment of anal fissure but which when administered together with the other active ingredient in the amount present in the composition is efficacious in the treatment of anal fissure.

39. A method of preparing an aqueous gel pharmaceutical composition comprising isosorbide dinitrate, the method comprising the step of dissolving the isosorbide dinitrate in dimethylsulfoxide to form a first solution and subsequently dissolving said first solution in a first aqueous solvent, whereby to form said aqueous gel.

40. A method according to claim 39, wherein said first aqueous solvent contains an additional pharmaceutically active agent dissolved therein.

41. A method according to claim 40, wherein said additional pharmaceutically active agent is a calcium channel blocker.

42. A method according to claim 41 wherein said calcium channel blocker is nifedipine.

43. A method according to claim 40, wherein said additional pharmaceutically active agent has been dissolved in a second aqueous solvent to form a second solution and said second solution has then been mixed into said first aqueous solvent prior to addition of said first solution thereto.

44. A method according to claim 39, wherein said first aqueous solvent comprises up to 15 wt.% of a water-soluble lipophilic substance dissolved therein.

45. A method according to claim 44 wherein said water-soluble lipophilic substance is selected from the group consisting of polyethylene glycol 400 and propylene glycol.

46. A method according to claim 39, wherein the isosorbide dinitrate is present at a concentration between about 0.1 and about 0.4 wt.% of the gel formed.

47. A method according to claim 46, wherein the isosorbide dinitrate is present at a concentration of about 0.2 wt.% of the gel formed.

48. A method according to claim 40, wherein the isosorbide dinitrate is present at a concentration between about 0.1 and about 0.4 wt.% of the gel formed, and said additional pharmaceutically acceptable agent is present at a concentration between about 0.1 and about 0.4 wt.% of the gel formed.

49. A method according to claim 48, wherein the isosorbide dinitrate is present at a concentration of about 0.2 wt.% of the gel formed, and said additional pharmaceutically acceptable agent is present at a concentration of about 0.2 wt.% of the gel formed.

50. A method according to claim 48, wherein said additional pharmaceutically active agent is nifedipine.

51. A method according to claim 49, wherein said additional pharmaceutically active agent is nifedipine.

52. A method for treating anal fissure, comprising applying to an affected locus of a patient suffering from anal fissure an efficacious amount of a combination of isosorbide dinitrate (ISDN) and a calcium channel blocker (CCB).

53. A method according to claim 52, wherein said ISDN and said CCB are provided in separate pharmaceutical preparations and are applied separately to said locus.

54. A method according to claim 53 wherein said calcium channel blocker is diltiazem.

55. A method according to claim 53 wherein said calcium channel blocker is nifedipine.

56. A method according to claim 53 wherein said ISDN and said CCB are applied simultaneously.

57. A method according to claim 53 wherein said ISDN and said CCB not applied simultaneously and an efficacious amount of each pharmaceutical preparation is applied to said locus.

58. A method according to claim 57 wherein said ISDN is applied prior to defecation and said CCB is applied two to four times a day without reference to defecation.

59. A method according to claim 53 wherein said separate pharmaceutical compositions are both gels.
